(19) United States
(12) Patent Application Publication
(10) Pub. No.: US 2023/0068593 A1
(43) Pub. Date: Mar. 2, 2023

(54) PROCESSES FOR PREPARING TUCATINIB POLYMORPHS

(71) Applicant: Macfarlan Smith Limited, Edinburgh (GB)

(72) Inventors: Thierry BONNAUD, Cambridge (GB); Adam Ross PATTERSON, Cambridge (GB); Zoe PRENTICE, Cambridge (GB); Mark Alexander WRIGHT, Cambridge (GB)

(21) Appl. No.: 17/815,400

(22) Filed: Jul. 27, 2022

Related U.S. Application Data
(60) Provisional application No. 63/203,545, filed on Jul. 27, 2021.

(51) Int. Cl. C07D 471/04

(52) U.S. Cl. CPC C07D 471/04; C07B 2200/13

(57) ABSTRACT

The present invention is directed to processes for making various polymorphs of tucatinib, designated herein as Form I, Form II, and Form III of tucatinib. These forms can then be used to prepare pharmaceutical formations containing tucatinib. Tucatinib is a tyrosine kinase inhibitor that is indicated for the treatment of breast cancer.

### FIELD OF THE INVENTION

The invention relates to processes for preparing various crystalline forms of tucatinib. These crystalline forms can then be used in preparing medicaments containing tucatinib.

### BACKGROUND OF THE INVENTION

Tucatinib, having the chemical designation, (N4-(4-([1,2,4]triazolo[1,5-a]pyridin-7-yloxy)-3-methylphenyl)-N6-(4,4-dimethyl-4,5-dihydrooxazol-2-yl)quinazoline-4,6-diamine, is a tyrosine kinase inhibitor of HER2. Tucatinib has the following structure:

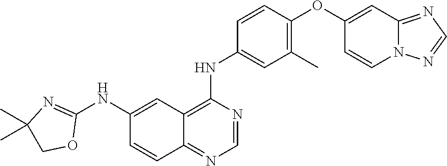 [SMILES: Cc1cc(Nc2ncnc3ccc(NC4=NC(C)(C)CO4)cc23)ccc1Oc1ccn2ncnc2c1]

Tucatinib is marketed under the tradename TUKYSA®. TUKYSA® is indicated in combination with trastuzumab and capecitabine for the treatment of adult patients with advanced unresectable or metastatic HER2-positive breast cancer, including patients with brain metastases, who have received one or more prior anti-HER2-based regimens in the metastatic setting.

U.S. Pat. No. 9,168,254 discloses Forms A-P of tucatinib and processes for their preparation.

### SUMMARY OF THE DISCLOSURE

The present invention is directed to processes of making crystalline forms of tucatinib, designated herein as Form I, Form II, and Form III of tucatinib.

### BRIEF DESCRIPTION OF THE DRAWINGS

FIG. 1 provides a representative XRPD pattern of a representative sample of Form I of tucatinib.

FIG. 2 provides a representative TGA thermogram of Form I of tucatinib.

FIG. 3 and FIG. 4 provide DSC thermograms of Form I of tucatinib.

FIG. 5 provides a representative GVS isotherm plot of Form I of tucatinib.

FIG. 6 provides a representative XRPD pattern of a representative sample of Form II of tucatinib.

FIG. 7 provides a TGA thermogram of Form II of tucatinib.

FIG. 8 and FIG. 9 provide DSC thermograms for Form II of tucatinib.

FIG. 10 provides a representative GVS isotherm plot of Form II of tucatinib.

FIG. 11 provides a representative XRPD pattern of a representative sample of Form III of tucatinib.

### DETAILED DESCRIPTION OF THE DISCLOSURE

The present disclosure is directed to processes of making various polymorphs of tucatinib, designated herein as Form I, Form II, and Form III of tucatinib.

As used herein and unless otherwise specified, the term “solid-state form” includes crystalline or polymorphic forms, amorphous phase, and solvates.

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, the terms “polymorph,” “polymorphic form” or related term herein, refer to a crystal form of an API (active pharmaceutical ingredient) free base or salt thereof that can exist in two or more forms, as a result of different arrangements or conformations of the molecule, ions of the salt, or addition and arrangement of solvents within the crystalline lattice.

As used herein and unless otherwise specified, the terms “substantially” or “substantially free/pure” with respect to a polymorph or polymorphic form means that the form contains about less than 30 percent, about less than 20 percent, about less than 15 percent, about less than 10 percent, about less than 5 percent, or about less than 1 percent by weight of impurities. Impurities may, for example, include other polymorphic forms, water and solvents other than that in a solvated crystalline polymorphic form.

Techniques for characterizing crystal and amorphous forms include but are not limited to differential scanning calorimetry (DSC), thermal gravimetric analysis (TGA), gravimetric vapor sorption (GVS), X-ray powder diffractometry (XRPD), single crystal X-ray diffraction (SCXRD), proton nuclear magnetic resonance (¹H-NMR), Fourier transform infrared spectroscopy (FTIR Spectroscopy), and Optical Microscopy.

XRPD data are collected using either a Bruker AXS D8 Advance diffractometer (used for Form I and Form II) or a PANalytical Empyrean diffractometer (used for Form III), using the methods set forth below.

### Bruker AXS D8 Advance

XRPD diffractograms are collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through a 8.0 mm receiving slit with 2.5° Soller slits followed by the Lynxeye Detector. The software used for data collection and analysis is Diffrac Plus XRD Commander and Diffrac Plus EVA respectively.
Samples are run under ambient conditions as flat plate specimens using powder as received. The sample is prepared on a polished, zero-background (510) silicon wafer by gently pressing onto the flat surface or packed into a cut cavity. The sample is rotated in its own plane.
The details of the data collection method are:
Angular range: 2 to 42° 2θ
Step size: 0.05° 2θ
   Collection time: 0.5 s/step (total collection time: 6.40 min)

### PANalytical Empyrean

XRPD diffractograms are collected on a PANalytical Empyrean diffractometer using Cu Kα radiation (45 kV, 40 mA) in transmission geometry. A 0.5° slit, 4 mm mask and 0.04 rad Soller slits with a focusing mirror are used on the incident beam. A PIXcel^{3D} detector, placed on the diffracted beam, is fitted with a receiving slit and 0.04 rad Soller slits. The software used for data collection is X'Pert Data Collector using X'Pert Operator Interface. The data are analysed and presented using Diffrac Plus EVA or HighScore Plus.
Samples are prepared and analysed in a metal 96 well-plate in transmission mode. X-ray transparent film is used between the metal sheets on the metal well-plate and powders (approximately 1-2 mg) are used as received.
The scan mode for the metal plate uses the gonio scan axis.
The details of the data collection method are:
Angular range: 2.5 to 32.0° 2θ
Step size: 0.0130° 2θ
   Collection time: 12.75 s/step (total collection time of 2.07 min)

### Thermo-Gravimetric Analysis (TGA)

TGA data are collected on a TA Instruments Q500 TGA, equipped with a 16 position auto-sampler. Typically, 5-10 mg of each sample is loaded onto a pre-tared aluminium DSC pan and heated at 10° C./min from ambient temperature to 350° C. A nitrogen purge at 60 ml/min is maintained over the sample.
The instrument control software is Advantage for Q Series and Thermal Advantage and the data are analysed using Universal Analysis.

### Differential Scanning Analysis (DSC)

DSC data are collected on a TA Instruments Q2000 equipped with a 50 position auto-sampler. Typically, 0.5-3 mg of each sample, in a pin-holed aluminium pan, is heated at 10° C./min from 25° C. to 300° C. A purge of dry nitrogen at 50 ml/min is maintained over the sample.

### Gravimetric Vapor Sorption (GVS)

Sorption isotherms are obtained using a Hiden IGASorp moisture sorption analyser, controlled by Isochema HISorp 2019 software (v4.02.0074). The sample temperature is maintained at 25° C. by a Grant LT ecocool 150 re-circulating water bath. The humidity is controlled by mixing streams of dry and wet nitrogen, with a total flow rate of 250 ml·min⁻¹. The relative humidity is measured by a calibrated Vaisala RH probe (dynamic range of 0-95% RH), located near the sample. The weight change, (mass relaxation) of the sample as a function of % RH is constantly monitored by the microbalance (accuracy ±0.001 mg).
Typically, 20-30 mg of sample is placed in a tared mesh stainless steel basket under ambient conditions. The sample is loaded and unloaded at 40% RH and 25° C. (typical room conditions). A moisture sorption isotherm is performed as outlined below (2 scans giving 1 complete cycle). The standard isotherm is performed at 25° C. at 10% RH intervals over a 0-90% RH range. Typically, a double cycle (4 scans) is carried out. Data analysis is carried out within the Isochema HISorp 2019 software and exported into Microsoft Excel to present accordingly.

### Method Parameters for Hiden IGASorp Experiments

| Parameters | Values |
|---|---|
| Adsorption-Scan 1 | 40-90 |
| Desorption/Adsorption-Scan 2 | 90-0, 0-40 |
| Intervals (% RH) | 10 |
| Number of Scans | 2 |
| Flow rate (ml · min⁻¹) | 250 |
| Temperature (° C.) | 25 |
| Stability (° C..min⁻¹) | 0.05 |
| Minimum Sorption Time (mins) | 10 |
| Maximum Sorption Time (mins) | 360 |
| Equilibration Mode | Final Rate |
| Accuracy (tolerance) | +/− 0.001 mg/min (over 600 s) |

Form I of tucatinib is prepared comprising:
a) combining tucatinib and DMSO at an elevated temperature to form a solution; and
b) adding water to the solution to yield Form I of tucatinib as a precipitate.
   In one embodiment, the volume of DMSO is not particularly limiting as long as a solution is formed. In one embodiment, about 2-4 volumes of DMSO (mL) is used per weight (g) of tucatinib. In one embodiment, about 2 volumes of DMSO is used. In another embodiment, about 4 volumes of DMSO is used. In one embodiment, the elevated temperature is about 50-70° C. In one embodiment, the elevated temperature is about 60° C. In another embodiment, the elevated temperature is about 65° C. In one embodiment, stirring is used during the combining. In one embodiment, the water is added to the solution in portions. In a particular embodiment, about 1 volume of water (mL) per weight (g) of tucatinib is added in each portion. In another embodiment, the water is added drop-wise. In one embodiment, about 4-10 volumes (mL) of water in total is added per weight (g) of tucatinib. In another embodiment, water is added until a DMSO/water ratio (v/v) of about 1:5 is obtained. The volume of water can be adjusted based on the level of precipitation that has occurred. One embodiment further comprises optionally adding seeds of Form I of tucatinib to the water forming a suspension before adding the water to the solution. The amount of seeds of Form I of tucatinib added to the water is not critical as long as a suspension is formed, indicating that the seeds of Form I of tucatinib are not completely dissolved in the water. It will also be apparent to one of ordinary skill in the art that if seeds of Form I of tucatinib are used, the elevated temperature should not be so high as to cause the seeds to completely dissolve or, alternatively, the temperature can be lowered before adding the suspension of seeds. In one embodiment, the amount of seeds added to the water is about 0.1-5 wt %, particularly about 1-2.5 wt %, of the tucatinib. In one embodiment, the seeds of Form I of tucatinib are added to a portion of the water to be added to the solution and the remaining water (without seeds) can then be added to the solution. In one embodiment, the seed suspension can be added in portions. In another embodiment, the seed suspension can be added drop-wise. One embodiment optionally comprises stirring after the water is added. In one embodiment, the stirring is for about 10 minutes to about 90 minutes. One embodiment optionally comprises cooling the mixture of step b) to about 20-30° C., particularly to about 25° C. Another embodiment further comprises isolating the precipitate. In one embodiment, the isolating is performed by filtration. In a particular embodiment, the isolating is performed by vacuum filtration. One embodiment optionally comprises washing the precipitate after filtration. In one embodiment, the precipitate is washed with acetonitrile. In one embodiment, about 3-5 volumes (mL), particularly about 4 volumes, of acetonitrile is used for the wash per weight (g) of tucatinib. In another embodiment, the precipitate is washed with water, followed by acetone. In one embodiment, about 1-3 volumes (mL), particularly about 2 volumes, of water is used for the wash per weight (g) of tucatinib. In one embodiment, about 1-3 volumes (mL), particularly about 1 volume, of acetone is used for the wash per weight (g) of tucatinib. One embodiment further comprises drying the precipitate. In one embodiment, the drying is under vacuum. In one embodiment, the drying is in a vacuum oven at about 40-50° C. and about 5 mbar. In one embodiment, the drying occurs for about 3-14 hours.

Form II of tucatinib is prepared comprising:
a) suspending tucatinib in a solvent mixture of DMSO and water;
b) heating the suspension to an elevated temperature to form a solution; and
c) cooling the solution to a lower temperature to yield Form II of tucatinib.
   In one embodiment, the volume of solvent mixture is not particularly limiting as long as a suspension is formed. In one embodiment, about 16-20 volumes, particularly about 18 volumes, of solvent mixture (mL) is used per weight (g) of tucatinib. In one embodiment, the volume ratio of DMSO to water in the solvent mixture is about 5-9:1, particularly about 5:1. One embodiment further comprises stirring the suspension at about 5-15° C., particularly at about 10° C., for about 15 minutes to 1 hour, particularly for about 30 minutes, before heating the suspension. In one embodiment, the elevated temperature is about 60-90° C., particularly about 80° C. In one embodiment, the heating rate is incremental. As will be apparent to one of ordinary skill in the art, the heating rate is not critical. In one embodiment, the heating rate is about 0.1° C. to about 1° C./minute. In a particular embodiment, the heating rate is about 0.5° C./minute. One embodiment further comprises stirring the solution at the elevated temperature before cooling. In one embodiment, the stirring is for about 5-20 minutes, particularly for about 10 minutes. One embodiment optionally comprises polish filtering the solution before cooling. In one embodiment, the cooling is to about 5-15° C., particularly to about 10° C. In one embodiment, the cooling rate is incremental. In one embodiment, the cooling rate is about 0.1° C. to about 1° C./minute. In a particular embodiment, the cooling rate is about 0.5° C./minute. Another embodiment further comprises isolating Form II of tucatinib. In one embodiment, the isolating is performed by filtration. In a particular embodiment, the isolating is performed by vacuum filtration. One embodiment optionally comprises washing Form II of tucatinib after filtration. In one embodiment, the Form II of tucatinib is washed with water, followed by acetone. In one embodiment, about 1-3 volumes (mL), particularly about 2 volumes, of water is used for the wash per weight (g) of tucatinib. In one embodiment, about 1-3 volumes, particularly about 1 volume, of acetone is used for the wash per weight (g) of tucatinib. In one embodiment, Form II of tucatinib is dried. Drying may be performed by any means knows to one of ordinary skill in the art, for example, use of a vacuum or vacuum oven.

In another embodiment of the invention, Form II of tucatinib is prepared comprising:
a) combining tucatinib and DMSO at an elevated temperature to form a solution;
b) cooling the solution to a first lower temperature;
c) optionally adding water to the solution, wherein step b) may be performed before or after step c);
d) adding seeds of Form II of tucatinib to the solution to form a suspension; and
e) adding additional water to the suspension to yield Form II of tucatinib, wherein the suspension is optionally cooled to a second lower temperature before or after the adding additional water.
   In one embodiment, the volume of DMSO is not particularly limiting as long as a solution is formed. In one embodiment, about 3-6 volumes of DMSO (mL), particularly about 5 volumes, are used per weight (g) of tucatinib. In one embodiment, stirring is used during the combining. In one embodiment, the solution is kept at the elevated temperature for a period of time. In one embodiment, the solution is kept at the elevated temperature for about 30-60 minutes, particularly for about 40 minutes. In one embodiment, the elevated temperature is about 50-70° C. In one embodiment, the elevated temperature is about 60° C. In another embodiment, the elevated temperature is about 65° C. In one embodiment, the first lower temperature is about 35-45° C., particularly about 40° C. In one embodiment, the cooling rate is incremental. In one embodiment, the cooling rate is about 0.1° C. to about 1° C./minute. In a particular embodiment, the cooling rate is about 1° C./minute. In one embodiment, the solution is maintained at the first lower temperature for a period of time, for example, for about 10 minutes. In one embodiment, the solution is stirred while maintained at the first lower temperature. In one embodiment, about 0.5-2 volumes (mL), particularly about 0.5-1 volume, of water is added to the solution. In one embodiment, the water is added dropwise. In one embodiment, the solution is stirred after the water is added. In one embodiment, step b) occurs before step c). In another embodiment, step c) occurs before step b). It will be apparent to one of ordinary skill in the art that water may be added to the solution before the seeds are added in order to decrease the solubility of the seeds in the solution. It will also be apparent to one of ordinary skill in the art that factors such as the volume of the DMSO and temperature will affect the solubility of the seeds in the solution. The amount of seeds of Form II of tucatinib is not critical as long as the seeds of Form II of tucatinib are not completely dissolved in the solution. In one embodiment, the amount of seeds of Form II of tucatinib is about 0.1-5 wt %, particularly about 2.5-5 wt %, of the tucatinib. In one embodiment, the seeds are suspended in about 5-10 volumes (mL), particularly about 10 volumes, of water per weight (g) of the seeds. In one embodiment, the suspension of seeds of Form II of tucatinib is added dropwise to the solution. Additional water may be used to facilitate transfer of the seed suspension to the solution. One embodiment further comprises stirring after the seeds are added. In one embodiment, about 3-5 volumes (mL), particularly about 4 volumes, of additional water are added to the mixture. In a particular embodiment, the additional water is added dropwise. In one embodiment, the additional water is added over about 5-20 minutes, particularly over about 10 minutes. In one embodiment, the mixture is stirred for about 15-60 minutes, particularly about 30 minutes, after the additional water is added. In one embodiment, the second lower temperature is about 20-30° C., particularly about 25° C. In one embodiment, the cooling rate is incremental. In one embodiment, the cooling rate is about 0.1° C. to about 1° C./minute. In a particular embodiment, the cooling rate is about 1° C./minute. Another embodiment further comprises isolating the Form II of tucatinib. In one embodiment, the isolating is performed by filtration. In a particular embodiment, the isolating is performed by vacuum filtration. One embodiment optionally comprises washing the Form II of tucatinib after filtration. In one embodiment, the Form II of tucatinib is washed with water, followed by acetone. In one embodiment, about 1-3 volumes (mL), particularly about 2 volumes, of water is used for the wash per weight (g) of tucatinib. In one embodiment, about 1-3 volumes, particularly about 1 volume, of acetone is used for the wash per weight (g) of tucatinib. One embodiment further comprises drying the Form II of tucatinib. In one embodiment, the drying is under vacuum. In one embodiment, the drying is in a vacuum oven at about 40-50° C. and about 5 mbar. One of skill in the art will appreciate that the drying time may vary, for example, depending on the quantity of material. In one embodiment, the drying occurs for about a few hours to a few days.

In another embodiment of the invention, Form II of tucatinib is prepared comprising:
a) combining tucatinib and DMSO at an elevated temperature to form a solution;
b) cooling the solution;
c) preparing a suspension seeds of Form II of tucatinib in water; and
d) adding the DMSO/tucatinib solution to the seed suspension to yield Form II of tucatinib as a precipitate.
   In one embodiment, the volume of DMSO is not particularly limiting as long as a solution is formed. In one embodiment, about 3-6 volumes of DMSO (mL), particularly about 4 volumes, are used per weight (g) of tucatinib. In one embodiment, stirring is used during the combining. In one embodiment, the elevated temperature is about 50-70° C., particularly about 60° C. In one embodiment, the cooling is to about 30-40° C., particularly to about 35° C. In one embodiment, the amount of seeds of Form II of tucatinib is not critical as long as the seeds of Form II of tucatinib form a suspension in the water. In one embodiment, the amount of seeds of Form II of tucatinib is about 0.1-5 wt %, particularly about 2.5-5 wt % of the tucatinib, more particularly about 5 wt %. In one embodiment, the seeds are suspended in about 70-90 volumes (mL), particularly about 80 volumes, of water per weight (g) of the seeds. In one embodiment, the suspension of seeds is stirred at room temperature. In one embodiment, the DMSO/tucatinib solution is added dropwise to the seed suspension. In one embodiment, the adding occurs over about 5-15 minutes, particularly over about 10 minutes. One embodiment further comprises stirring the mixture of DMSO/tucatinib solution and seed suspension. In one embodiment, the stirring is at room temperature. In another embodiment, the stirring is for about 5-15 minutes, particularly for about 10 minutes. Another embodiment further comprises isolating the precipitate. In one embodiment, the isolating is performed by filtration. In a particular embodiment, the isolating is performed by vacuum filtration. One embodiment optionally comprises washing the precipitate after filtration. In one embodiment, the precipitate is washed with water, followed by acetone. In one embodiment, about 1-3 volumes (mL), particularly about 1 volume, of water is used for the wash per weight (g) of tucatinib. In one embodiment, about 0.5-2 volumes (mL), particularly about 0.5 volume, of acetone is used for the wash per weight (g) of tucatinib. One embodiment further comprises drying the precipitate. In one embodiment, the drying is under vacuum. In a further embodiment, the drying is in a vacuum oven at about 40-50° C. and about 5 mbar. One of skill in the art will appreciate that the drying time may vary, for example, depending on the quantity of material and the amount of liquid remaining in the precipitate. In one embodiment, the drying occurs for about 5-10 hours, particularly for about 6 hours.

Form III of tucatinib is prepared comprising:
a) combining tucatinib and a solvent mixture of THF and water at an elevated temperature to form a solution;
b) cooling the solution; and
c) evaporating a portion of the solution to yield Form III of tucatinib as a precipitate.
   In one embodiment, the volume of solvent mixture is not particularly limiting as long as a solution is formed. In one embodiment, about 15-20 volumes (mL), particularly about 17.5 volumes, of the solvent mixture are used per weight (g) of tucatinib. In one embodiment, the volume ratio of THF to water in the solvent mixture is about 2-10:1, particularly about 9:1. In one embodiment, the elevated temperature is about 50-70° C., particularly about 60° C. In one embodiment, stirring is used during the combining. In one embodiment, the solution is cooled to a temperature lower than the elevated temperature. In a particular embodiment, the solution is cooled to about 35-45° C., particularly to about 40° C. In one embodiment, the cooling is incremental. In one embodiment, the cooling rate is about 0.1° C. to about 1° C./minute. In one embodiment, the portion of the solution that is evaporated is an amount that is sufficient to allow precipitation to occur. In a particular embodiment, about ½ to ¾ of the starting volume of the solvent mixture is evaporated, particularly about ¾. Any means known to one of skill in the art may be used for the evaporation. Some means include use of a rotary evaporator and/or heat. Another embodiment further comprises isolating the precipitate. In one embodiment, the isolating is performed by filtration. In a particular embodiment, the isolating is performed by vacuum filtration. One embodiment further comprises drying the precipitate. In one embodiment, the drying is under vacuum.

### EXAMPLES

Examples 1-10, which follow herein, disclose the preparation of Form I, Form II, and Form III of tucatinib.

The Examples are presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are illustrative of the present disclosure and the disclosure is not intended to be limited to the examples described herein and shown.

### Example 1—Preparation of Form I of Tucatinib

Tucatinib (25 mg) is dissolved in DMSO (50 μl, 2 volumes) at 60° C. Water (25 μl, 1 volume) is added sequentially until a final ratio of DMSO/water (1:5 v/v) is achieved, resulting in the formation of a precipitate. The suspension is filtered over a frit under gentle positive pressure to remove excess solvent. The isolated solid is identified as Form I of tucatinib.

XRPD 2θ pattern peaks and relative % intensity values for the peaks of Form I of tucatinib are shown in Table I.

TABLE 1
Average Peak List for Form I of tucatinib

| Angle | Intensity |
|---|---|
| (° 2θ) | (%) |
| 7.0 | 37.8 |
| 8.6 | 9.1 |
| 10.3 | 3.9 |
| 11.1 | 14.8 |
| 11.9 | 31.0 |
| 12.2 | 6.1 |
| 12.9 | 13.0 |
| 13.4 | 7.0 |
| 14.1 | 42.3 |
| 14.9 | 11.3 |
| 15.6 | 3.9 |
| 15.9 | 66.6 |
| 17.1 | 17.2 |
| 17.3 | 39.4 |
| 17.5 | 11.5 |
| 17.7 | 60.2 |
| 18.1 | 43.6 |
| 18.6 | 5.4 |
| 18.8 | 11.1 |
| 19.1 | 45.3 |
| 19.6 | 4.5 |
| 21.0 | 4.0 |
| 21.2 | 5.7 |
| 21.4 | 12.0 |
| 21.9 | 6.8 |
| 22.3 | 6.6 |
| 22.9 | 100.0 |
| 23.1 | 86.5 |
| 23.5 | 7.5 |
| 24.0 | 7.0 |
| 24.3 | 11.1 |
| 24.6 | 12.1 |
| 25.1 | 4.2 |
| 25.4 | 5.4 |
| 25.8 | 49.7 |
| 26.4 | 4.7 |
| 26.8 | 3.4 |
| 27.2 | 30.2 |
| 27.8 | 5.1 |
| 28.4 | 7.6 |
| 28.9 | 7.6 |
| 29.3 | 6.2 |
| 29.6 | 9.0 |
| 30.0 | 6.6 |
| 30.6 | 7.5 |

The angle measurements are ±0.2° 2⊖. Key defining peaks for solid-state Form I of tucatinib include peaks at 15.9, 17.7, 22.9, and 23.1° 2⊖.

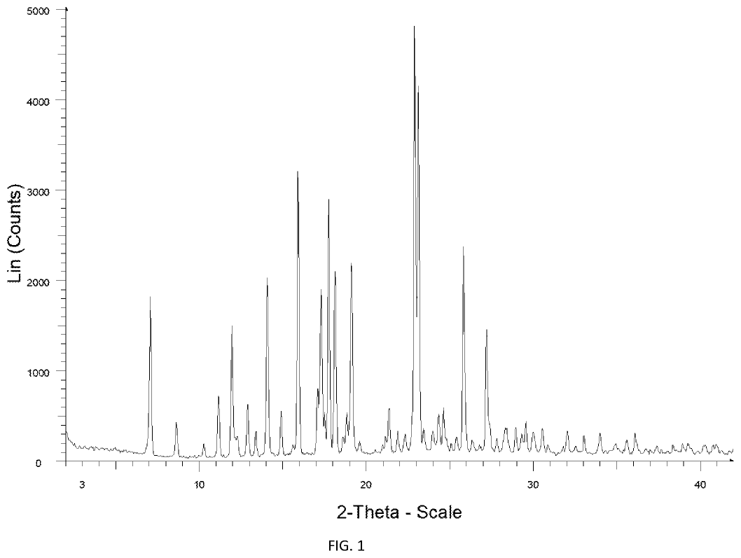

An XPRD pattern for a representative sample of Form I of tucatinib is shown in FIG. 1.

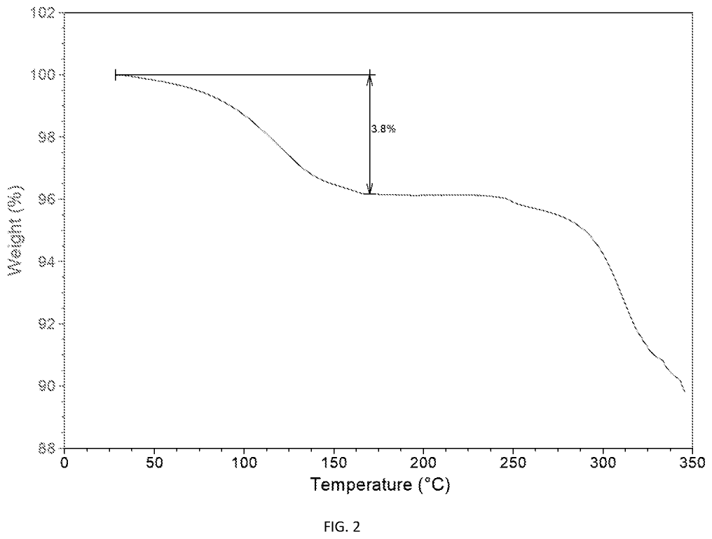

TGA analysis shows a loss of about 3.5-3.8 weight % up to about 160° C. A representative TGA thermogram of Form I of tucatinib is depicted in FIG. 2.

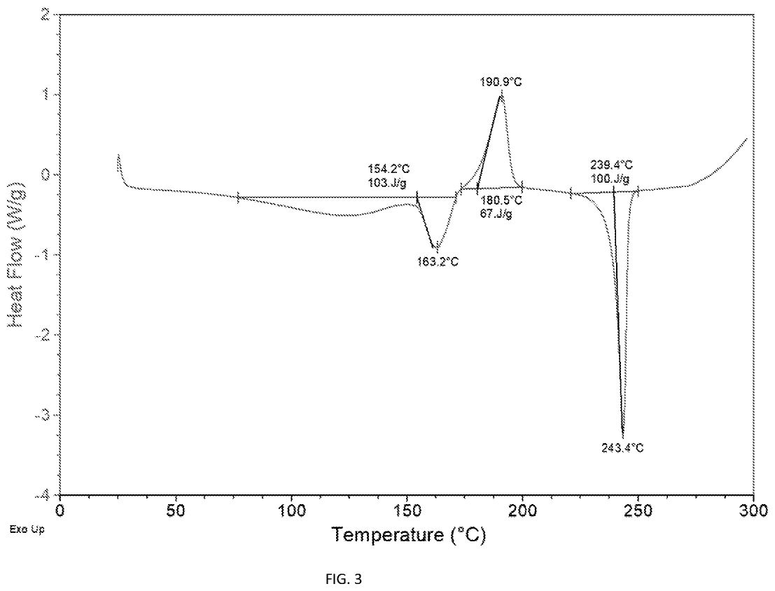

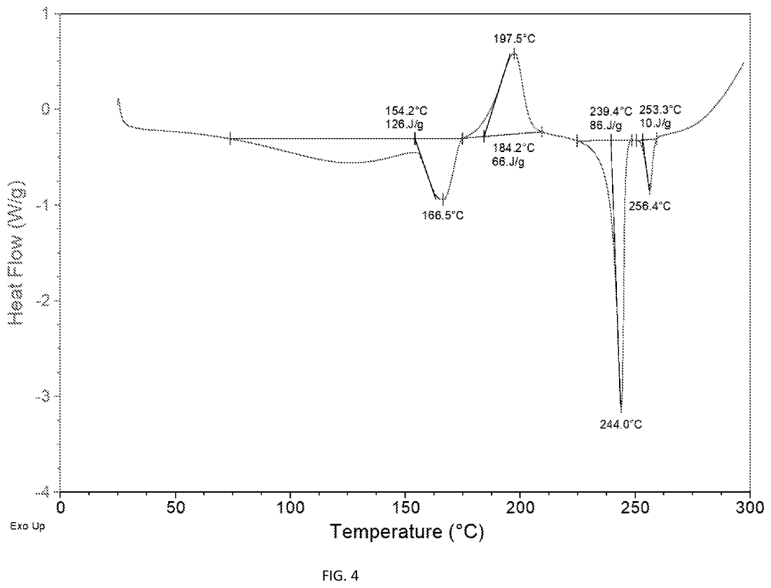

DSC analysis of Form I of tucatinib shows the onset of endothermic events at about 154° C. and 239° C., and a possible third endothermic event with an onset at about 253° C., and as depicted in FIG. 3 and FIG. 4. Without wishing to be bound, it is believed that Form I of tucatinib may convert to multiple anhydrous forms upon heating resulting in differing DSC thermograms.

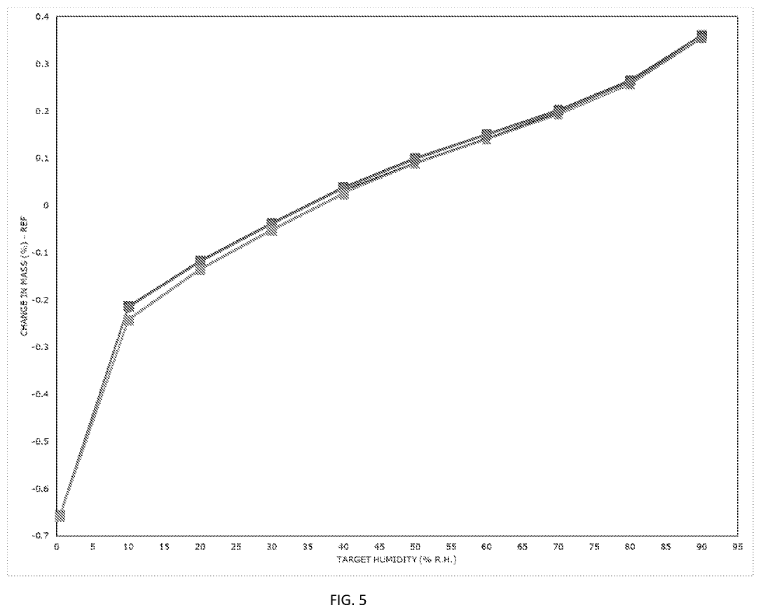

A representative GVS isotherm plot of Form I of tucatinib is depicted in FIG. 5.

### Example 2—Preparation of Form I of Tucatinib

Tucatinib (500 mg) is dissolved in DMSO (1.0 ml, 2 volumes) at 60° C. with stirring. Seeds of Form I of tucatinib prepared in Example 1 are suspended in water (2 ml, 4 volumes). The water suspension is slowly added to the DMSO solution. A suspension forms on addition and after 10 minutes stirring is isolated by Buchner filtration (Grade 1 Whatman filter) and dried in a vacuum oven for 3 hours (50° C., ˜5 mbar). The isolated solid is identified as Form I of tucatinib.

### Example 3—Preparation of Form I of Tucatinib

Tucatinib (500 mg) is dissolved in DMSO (1.0 ml, 2 volumes) at 60° C. with stirring (400 rpm). Seeds of Form I of tucatinib (˜5 mg) prepared in Example 2 are suspended in water (4.0 ml, 8 volumes) and added dropwise to the hot DMSO solution. Precipitation occurs on the water addition. Stirring is increased to 600 rpm and stirred for 90 minutes at 60° C. The resulting suspension is vacuum filtered and the filter cake is washed with acetonitrile (2.0 ml) and dried under vacuum for 15 minutes. The recovered material is dried in a vacuum oven at 50° C. (˜5 mbar) for ˜4 hours. The isolated material is determined to be Form I of tucatinib.

### Example 4—Preparation of Form I of Tucatinib

Tucatinib (1.00 g) is dissolved in DMSO (4.0 ml, 4 volumes) with stirring (500 rpm) at 65° C. The solution is cooled to 60° C. and seeds of Form I of tucatinib (25 mg, 2.5 wt %) are suspended in water (1.0 ml) and added dropwise. Precipitation occurs during addition. Additional water (3.0 ml) is added dropwise at 60° C. and results in additional precipitation. The mixture is cooled to 25° C. and vacuum filtered. The filter cake is washed with water (2 ml) and then acetone (1.0 ml) and dried under vacuum for 15 minutes. The recovered material is dried in a vacuum oven at 40° C. (˜5 mbar) for ˜14 hours. The isolated material is determined to be Form I of tucatinib.

### Example 5—Preparation of Form II of Tucatinib

Tucatinib (55 mg) is suspended in DMSO/water (5:1 v/v) and stirred (500 rpm) at 10° C. for 30 minutes. The mixture is heated to 80° C. at 0.5° C./minute and stirred isothermally at 80° C. for 10 minutes, and then cooled to 10° C. at 0.5° C./minute. The suspension is filtered over a frit under gentle positive pressure to remove excess solvent. The isolated solid is identified as Form II of tucatinib.

XRPD 2θ pattern peaks and relative % intensity values for the peaks of Form II of tucatinib are shown in Table 2.

TABLE 2
Average Peak List for Form II of tucatinib

| Angle | Intensity |
|---|---|
| (° 2θ) | (%) |
| 7.0 | 7.4 |
| 10.0 | 59.2 |
| 11.1 | 28.5 |
| 11.5 | 2.2 |
| 12.5 | 2.2 |
| 13.1 | 6.6 |
| 13.8 | 23.6 |
| 14.6 | 12.7 |
| 15.4 | 15.5 |
| 15.6 | 44.6 |
| 16.5 | 11.5 |
| 17.7 | 30.3 |
| 17.8 | 14.9 |
| 18.6 | 7.0 |
| 18.8 | 7.6 |
| 19.9 | 13.1 |
| 20.5 | 24.4 |
| 21.7 | 4.4 |
| 22.1 | 14.9 |
| 22.7 | 3.5 |
| 23.0 | 14.5 |
| 23.5 | 100.0 |
| 24.3 | 4.2 |
| 24.8 | 18.3 |
| 25.4 | 8.9 |
| 26.3 | 2.3 |
| 26.8 | 10.9 |
| 27.0 | 11.8 |
| 27.6 | 12.2 |
| 28.0 | 4.7 |
| 28.4 | 7.8 |
| 29.0 | 7.8 |
| 29.9 | 11.1 |

The angle measurements are ±0.2° 2⊖. Key defining peaks for solid-state Form II of tucatinib include peaks at 10.0, 15.6, 17.7, and 23.5° 2⊖.

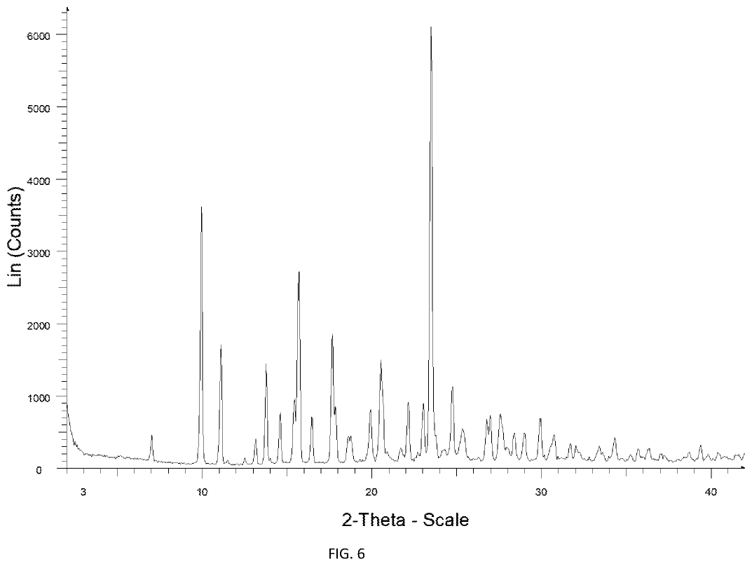

An XPRD pattern for a representative sample of Form II of tucatinib is shown in FIG. 6.

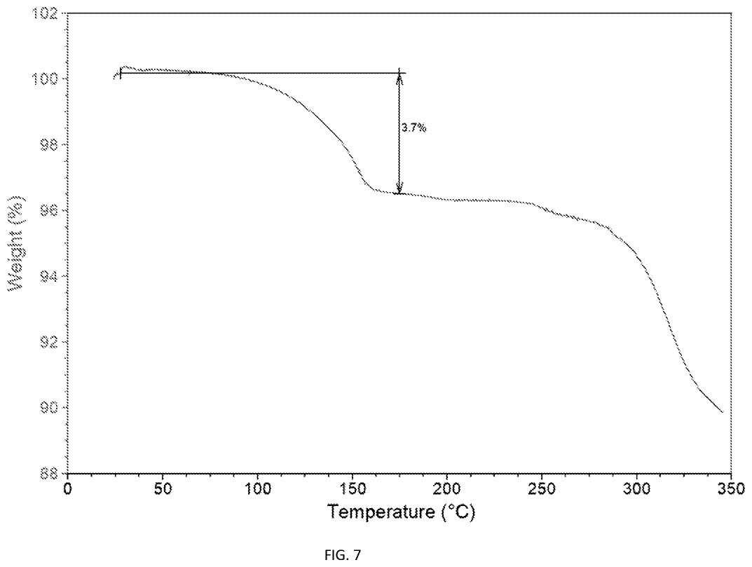

TGA analysis shows a loss of about 3.7-5.0 weight % up to about 165-175° C. A representative TGA thermogram of Form II of tucatinib is depicted in FIG. 7.

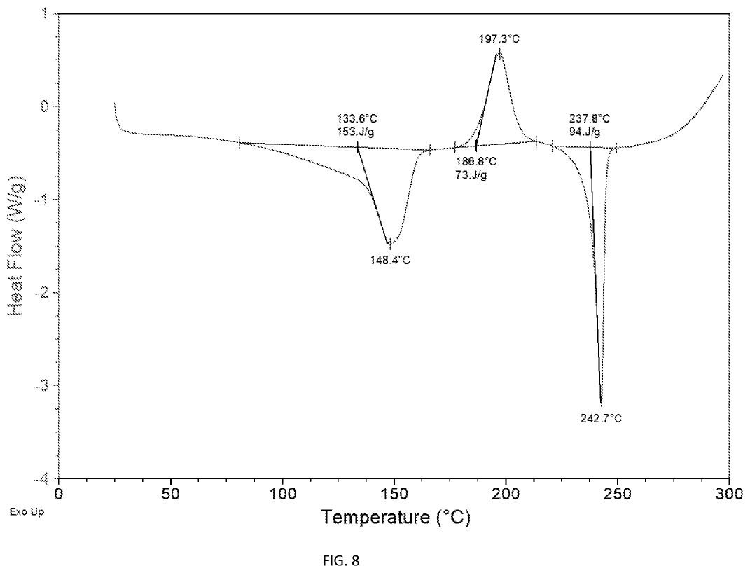

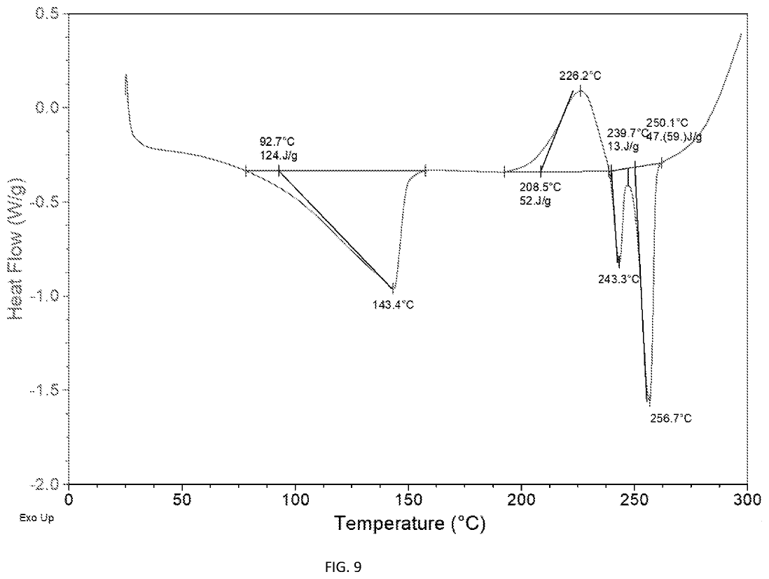

DSC analysis of Form II of tucatinib shows the onset of endothermic events at about 134° C. and 238° C. or at about 93° C., 240° C., and 250° C., as depicted in FIG. 8 and FIG. 9. Without wishing to be bound, it is believed that Form II of tucatinib may convert to multiple anhydrous forms upon heating resulting in differing DSC thermograms.

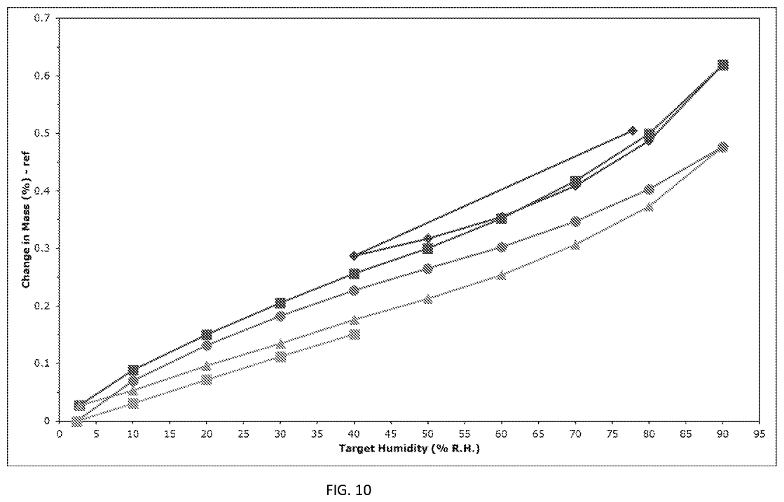

A representative GVS isotherm plot of Form II of tucatinib is depicted in FIG. 10.

### Example 6—Preparation of Form II of Tucatinib

Tucatinib (1.00 g) is suspended in DMSO/water (9:1 v/v) (10.0 ml, 10 volumes) and dissolved at 65° C. with stirring (400 rpm). The solution is cooled to 50° C. The solution is seeded with Form I of tucatinib (˜5 mg) prepared in Example 3 and the seeds dissolve. Water (˜1.5 ml) is added dropwise until sample remains turbid. The mixture is seeded with Form I of tucatinib (˜5 mg) and the seeds persist. A suspension develops over ˜5 minutes. Further water (˜3.5 ml) is added dropwise. A total of 5.0 ml (5 volumes) water is added. The mixture is cooled to 25° C. at 0.5° C./minute and stirred at the isolation temperature overnight. The suspension is vacuum filtered. The filter cake is washed with water (2.0 ml) followed by acetone (1.0 ml) and dried under vacuum for (15 minutes). The material is dried in a vacuum oven at 40° C. (˜5 mbar) for 2 hours. The isolated material is determined to be Form II of tucatinib.

### Example 7—Preparation of Form II of Tucatinib

Tucatinib (5.00 g) is dissolved in DMSO (25.0 ml, 5 volumes) at 65° C. The solution is cooled to 60° C. and water (5.0 ml, ˜15% v/v) is added dropwise. Some precipitation is observed on contact which redissolves. A yellow solution forms by the end of the addition. The solution is cooled to 40° C. at 1° C./min. The solution becomes turbid at ˜56° C. At 40° C., and the mixture is seeded with Form II of tucatinib (250 mg, 2.5 wt %) prepared in Example 6. The mixture is cooled to 25° C. at 1° C./min. Water (20.0 ml, 4 volumes) is added dropwise. The mixture is filtered, washed with water (10 ml), then acetone (5 ml), and the filter cake is dried under vacuum for 15 minutes. The recovered solid is placed in vacuum oven (40° C., 5 mbar) to dry for 3 days. The isolated material is determined to be Form II of tucatinib.

### Example 8—Preparation of Form II of Tucatinib

Tucatinib (5.00 g) is dissolved in DMSO (25.0 ml, 5 volumes) at 60° C. with stirring (150 rpm) and held at that temperature for ˜40 minutes. The solution is cooled to 40° C. at 1° C./minute and kept at held at that temperature for ˜10 minutes. A yellow solution persists. The stirring is increased to 300 rpm and water (2.5 ml, ˜9 vol %) is added. Some initial precipitation may occur which redissolves. After stirring, a yellow solution persists. Seeds of Form II of tucatinib (250 mg, 5 wt %) prepared in Example 7 are suspended in water (2.5 ml) and added dropwise to the DMSO solution. The seeds appear to take. A further 1.0 ml water aliquot is used to transfer residual seed material. The mixture is stirred for 10 minutes and a suspension forms. The suspension is cooled to 25° C. at 1° C./minute. At 25° C., further water (21.5 ml) is added dropwise over 10 minutes. The mixture is stirred for 30 minutes. The suspension is filtered under vacuum and the filter cake washed with water (10 ml, 2 volumes) and then acetone (5 ml, 1 volume). The filter cake is dried under vacuum for 15 minutes. The recovered solid is dried in a vacuum oven at 40° C. (˜5 mbar) for ˜17 hours. The isolated solid is determined to be Form II of tucatinib.

### Example 9—Preparation of Form II of Tucatinib

Tucatinib (1.00 g) is dissolved in DMSO (4.0 ml, 4 volumes) at 60° C. with stirring (500 rpm). The solution is cooled to 35° C. Seeds of Form II of tucatinib (50 mg, 5 wt %) prepared in Example 8 are suspended in water (4.0 ml) and stirred at room temperature. The DMSO solution is added dropwise to the water suspension over 10 minutes. Precipitation is observed on addition and a suspension results after the final addition. The suspension is stirred at room temperature for 10 minutes. The suspension is filtered under vacuum and the filter cake washed with water (1.0 ml) and then acetone (0.5 ml). The filter cake is dried under vacuum for ˜30 minutes. The recovered solid is dried in a vacuum oven at 40° C. (˜5 mbar) for ˜6 hours. The isolated solid is determined to be Form II of tucatinib.

### Example 10—Preparation of Form III of Tucatinib

Tucatinib (25 mg) is dissolved in THF/water (9:1 v/v) (437.5 μL, 17.5 volumes) at 60° C. with stirring (400 rpm). The solution is cooled to 40° C. and the solution evaporated to approximately one quarter of the starting volume of solvent. The resulting suspension is filtered over a frit under gentle positive pressure to remove excess solvent. The solid is isolated and determined to be Form III of tucatinib.

XRPD 2θ pattern peaks and relative % intensity values for the peaks of Form III of tucatinib are shown in Table 3.

TABLE 3
Average Peak List for Form III of tucatinib

| Angle | Intensity |
|---|---|
| (° 2θ) | (%) |
| 6.9 | 72.5 |
| 9.2 | 20.3 |
| 9.5 | 24.3 |
| 9.8 | 26.1 |
| 10.7 | 32.1 |
| 11.0 | 31.4 |
| 11.5 | 17.1 |
| 12.8 | 68.7 |
| 13.7 | 36.1 |
| 14.7 | 14.7 |
| 15.3 | 33.2 |
| 15.5 | 17.6 |
| 16.4 | 47.2 |
| 17.0 | 55.7 |
| 17.7 | 29.3 |
| 18.4 | 38.2 |
| 19.2 | 65.8 |
| 19.5 | 30.5 |
| 20.3 | 100.0 |
| 21.0 | 32.1 |
| 21.2 | 47.9 |
| 21.9 | 86.2 |
| 22.2 | 25.1 |
| 22.7 | 18.0 |
| 23.2 | 86.4 |
| 24.0 | 35.8 |
| 24.6 | 38.0 |
| 25.3 | 55.9 |
| 26.0 | 27.8 |
| 26.2 | 30.8 |
| 26.9 | 20.9 |
| 27.3 | 35.4 |
| 27.8 | 25.5 |
| 28.4 | 15.1 |
| 29.4 | 19.1 |
| 30.7 | 14.3 |
| 31.3 | 17.2 |

The angle measurements are ±0.2° 2⊖. Key defining peaks for solid-state Form III of tucatinib includes peaks at 20.3, 21.9, and 23.2° 2⊖.

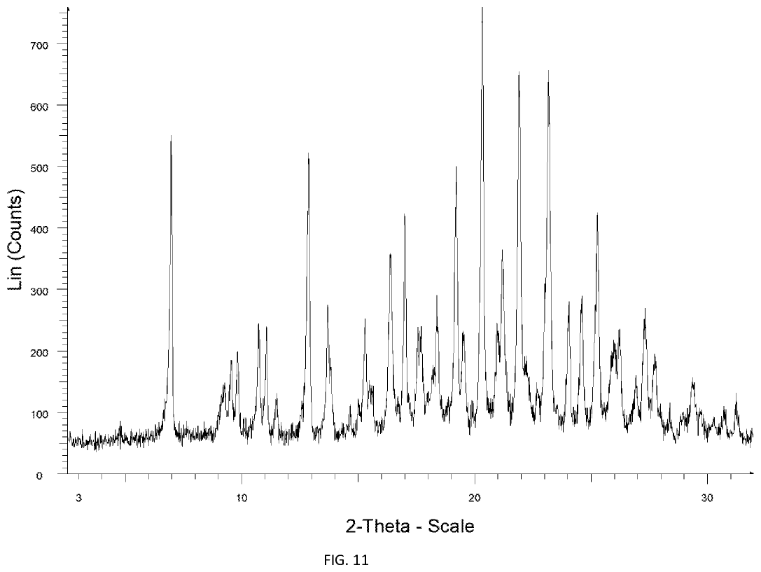

An XPRD pattern for a representative sample of Form III of tucatinib is shown in FIG. 11.

The above examples are set forth to aid in the understanding of the disclosure and are not intended and should not be construed to limit in any way the disclosure set forth in the claims which follow hereafter.

1. A process for the preparation of Form I of tucatinib comprising:
a) combining tucatinib and DMSO and heating to about 50-70° C. to form a solution; and
b) adding water to the solution to yield Form I of tucatinib as a precipitate, wherein about 2-4 volumes of DMSO (mL) is used per weight (g) of tucatinib.

2. The process of claim 1, further comprising adding seeds of Form I of tucatinib to the water forming a suspension before adding the water to the solution.

3. The process of claim 1, further comprising cooling the mixture of step b) to about 20-30° C.

4. A process for the preparation of Form II of tucatinib comprising:
a) suspending tucatinib in a solvent mixture of DMSO and water;
b) heating the suspension to about 70-90° C. at about 0.5° C./minute; and
c) cooling the suspension to about 5-15° C. at about 0.5° C./minute to yield Form II of tucatinib, wherein about 16-20 volumes of solvent mixture (mL) is used per weight (g) of tucatinib.

5. The process of claim 4, wherein the volume ratio of DMSO to water in the solvent mixture is about 5-9:1.

6. The process of claim 4, further comprising stirring the suspension at about 5-15° C. before heating the suspension.

7. The process of claim 4, further comprising stirring the suspension at the elevated temperature before cooling.

8. A process for the preparation of Form II of tucatinib comprising:
a) combining tucatinib and DMSO at about 50-70° C. to form a solution;
b) cooling the solution to a first lower temperature;
c) optionally adding water to the solution, wherein step b) may be performed before or after step c);
d) adding seeds of Form II of tucatinib to the solution to form a suspension;
e) adding additional water to the suspension to yield Form II of tucatinib, wherein the suspension is optionally cooled to a second lower temperature before or after the adding additional water and wherein about 3-6 volumes of DMSO (mL) are used per weight (g) of tucatinib.

9. The process of claim 8, wherein the solution is maintained at the elevated temperature for about 3-60 minutes before cooling.

10. The process of claim 8, wherein the first lower temperature is about 35-45° C. and wherein the cooling rate of step b) and the optional cooling step is about 1° C./minute.

11. The process of claim 8, wherein the amount of seeds of Form II of tucatinib is about 0.1-5 wt % of the tucatinib.

12. The process of claim 8, wherein the seeds are suspended in about 5-10 volumes (mL) of water per weight (g) of the seeds.

13. The process of claim 8, wherein the suspension of seeds of Form II of tucatinib is added dropwise to the solution.

14. The process of claim 17, wherein the second lower temperature is about 20-30° C.

15. A process for the preparation of Form II of tucatinib comprising:
a) combining tucatinib and DMSO at about 50-70° C. to form a solution;
b) cooling the solution;
c) preparing a suspension seeds of Form II of tucatinib in water; and
d) adding the DMSO/tucatinib solution to the seed suspension to yield Form II of tucatinib as a precipitate, wherein about 3-6 volumes of DMSO (mL) is used per weight (g) of tucatinib.

16. The process of claim 15, wherein the amount of seeds of Form II of tucatinib is about 0.1-5 wt % of the tucatinib.

17. The process of claim 15, wherein the seeds are suspended in about 70-90 volumes (mL) of water per weight (g) of the seeds wherein the DMSO/tucatinib solution is added dropwise to the seed suspension.

18. A process for the preparation of Form III of tucatinib comprising:
a) combining tucatinib and a solvent mixture of THF and water, then heating the mixture to about 50-70° C. to form a solution;
b) cooling the solution to about 35-45° C.; and
c) evaporating a portion of the solution to yield Form III of tucatinib as a precipitate, wherein about 15-20 volumes (mL) of the solvent mixture is used per weight (g) of tucatinib and wherein the volume ratio of THF to water in the solvent mixture is about 9:1.

19. The process of claim 18, wherein the portion of the solution that is evaporated is about ½ to ¾ of the starting volume of the solvent mixture.
